(19) United States
(12) Patent Application Publication
(10) Pub. No.: US 2011/0197288 A1
(43) Pub. Date: Aug. 11, 2011

(54) Transgenic animal for screening of compounds that modulate cell proliferation, and its use in the pharmaceutical field

(76) Inventors: Adriana Maggi, Milano (IT); Paolo Ciana, Gravellona Toce (IT); Giulia Piaggio, Roma (IT); Frauke Goeman, Roma (IT); Ada Sacchi, Roma (IT); Cecilia Tiveron, Roma (IT)

(21) Appl. No.: 12/926,435

(22) Filed: Nov. 17, 2010

(30) Foreign Application Priority Data
Nov. 17, 2009 (IT) MI2009A002023

(51) Int. Cl. G01N 33/50; A01K 67/027; G01N 33/00

(52) U.S. Cl. 800/3; 800/14; 800/18; 435/7.21

(57) ABSTRACT

The present invention relates to a transgenic non-human mammalian animal whose genome incorporates a biosensor of the activity of modulators of cell proliferation consisting of a DNA sequence that comprises the luciferase reporter transgene ligated downstream of a promoter sequence of the CYCB2 gene, this sequence promoter/reporter being flanked by HS4 insulator sequences at each of its 3′ and 5′ sides.

The transgenic animal is proposed for the screening of compounds with pharmacologically relevant activities.

The invention provides for a further use of the animal line for the study and toxicological evaluation of compounds with tumorigenic activity, and also for the study and evaluation of compounds with chemopreventive anti-cancer activity.

This is application claims the priority of Italian number MI2009A002023 filed Nov. 17, 2009, hereby incorporated by reference.

### DESCRIPTION

The present invention relates to a transgenic animal for the screening of compounds endowed with activities of pharmacological interest, and to its use in the pharmaceutical field.

A dysregulation of signal transduction pathways involved in cell cycle progression through different phases, resulting in hyper-or hypo-proliferation, is at the basis of several human diseases. For this reason, active compounds on cell proliferation are the subject of great pharmacological interest.

Currently there are no known systems that make possible to measure in a simple, direct and ubiquitous way, and in all tissues of an animal model, the activity of compounds able to modulate cell proliferation. Instead it would be desirable to obtain the activity profile of the study compounds both in living animals, especially by application of in vivo non-invasive imaging techniques, and in any kind of explanted tissue by ex vivo imaging techniques or biochemical determination of enzymatic activities.

Currently most systems used for pre-clinical screening of compounds with anti- or pro-proliferative activity are based both on cell culture models and on transplants in immunodeficient animals.

Instead the evaluation of proliferative activity in normal or tumor tissues in an animal model provides for the histochemical determination of endogenously expressed proliferation markers, such as the nuclear protein Ki67, or the administration of markers, such as bromodeoxyuridine. In this case the analysis, in addition to being laborious and involving the sacrifice of the animal, does not allow an easy quantification of proliferative activity. A method of analysis by in vivo imaging or by biochemical analysis of a reporter activity would be a good alternative because it is rapid, inexpensive, applicable to living animals, thus allowing in all cases a quantitative measurement of the pharmacological activity under study.

U.S. 7041869 proposed a transgenic mouse model that can be used to monitor, by in vivo imaging, the areas of tumor growth where bioluminescence is produced by the luciferase (Luc) reporter gene driven by E2F-1 promoter, acting as cell cycle biosensor. Use of this transgenic mouse model for drug screening is limited by the fact that, on the one hand, the analytical application is restricted to tumor tissues and, on the other hand, the biosensor constructed in this way cannot measure accurately the proliferative state in all tissues of the animal in an ubiquitous manner. In fact, a biosensor such as the E2F-Luc reporter transgene can only detect proliferation of specific cancer cells carrying an inactive Rb tumor suppressor.

It is instead desired a drug screening method that overcomes these limitations and allows measurement of the proliferative status of all cells, hence of the activity of the compound under investigation on cell proliferation in all tissues, both normal and cancerous.

To achieve this goal, the present invention proposes a mammalian non-human transgenic animal whose genome incorporates, as a biosensor of the activity of cell proliferation modulators, a DNA sequence comprising the luciferase reporter transgene ligated downstream of the sequence of the CYCB2 gene promoter, where the whole promoter/reporter sequence is flanked by HS4 insulator sequences at both 3′ and 5′ sides.

In a preferred embodiment, the transgenic animal according to the invention is a mouse.

The characterization of the sequences mentioned above is reported below, including the GenBank accession number:
CYCB2, Mus musculus, GenBank: AY788998.1—Reference: Lange-zu D. et al., FEBS Lett. 484 (2), 77-81 (2000)
Luciferase, Photinus pyralis, GenBank: M15077.1—Reference: de Wet, J. R., Mol. Cell. Biol. 7 (2), 725-737 (1987)
HS4 insulator, beta-globin gene Gallus gallus, GenBank: AJ277959—Reference: Ciana P., Mol. Endocrinol. 15 (7), 1104-1113 (2001), corresponding to SEQ ID NO:4.

The invention provides for a first use of a transgenic non-human mammalian animal in order to study and develop compounds of potential pharmacological interest with inhibitory activity on cell proliferation. In this context, the animal is expected to be used especially to investigate and develop compounds with antitumor activity and compounds with anti-inflammatory activity.

The invention involves a further use of the animal to investigate and develop compounds of potential pharmacological interest with cell proliferation inducing activity. In particular, in this context, it is envisaged its use to study and develop compounds of potential pharmacological interest such as growth factors that selectively stimulate stem cells proliferation.

The invention provides for a further use of the transgenic non-human mammalian animal for the study and toxicological evaluation of compounds with tumorigenic activity.

The invention provides for a further use of the transgenic non-human mammalian animal for the study and evaluation of compounds with anti-cancer chemopreventive activity.

The invention relates also to a method for evaluation of the above discussed activities, based on the generation of a transgenic non-human mammalian animal wherein cell proliferation can be simultaneously monitored in all normal or tumor tissues. The method is generally characterized by the following steps:
Generate a construct consisting of a nucleotide sequence comprising a luciferase reporter transgene ligated downstream of a specific sequence of the CYCB2 gene promoter, in which both 3′ and 5′ sides of the promoter/reporter sequence are flanked by HS4 insulator sequences;
Incorporate this construct into the genome of the animal;
Treat individuals of this transgenic animal line with said modulator of cell proliferation;
Assess the amount of luciferase produced in vivo by imaging assays performed on the animal or ex vivo by enzyme activity assays performed on tissues and cells;
Compare the luminescence emission of said individual animals treated with said modulator of cell proliferation with the other untreated individuals of the same transgenic animal line.

The following experimental part, in reference to the enclosed figures, is described for a better understanding of the characteristics and advantages of the invention and should not be intended in any way to limit the scope of the invention itself.

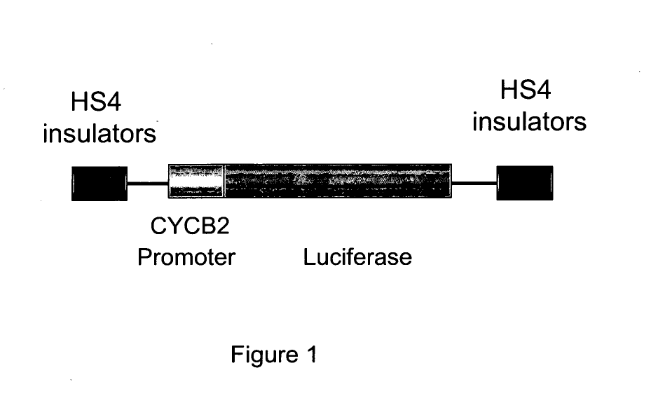

FIG. 1 schematically shows the luciferase reporter transgene construct, comprising the HS4 insulator sequences flanking said luciferase reporter transgene ligated to the CYCB2 promoter.

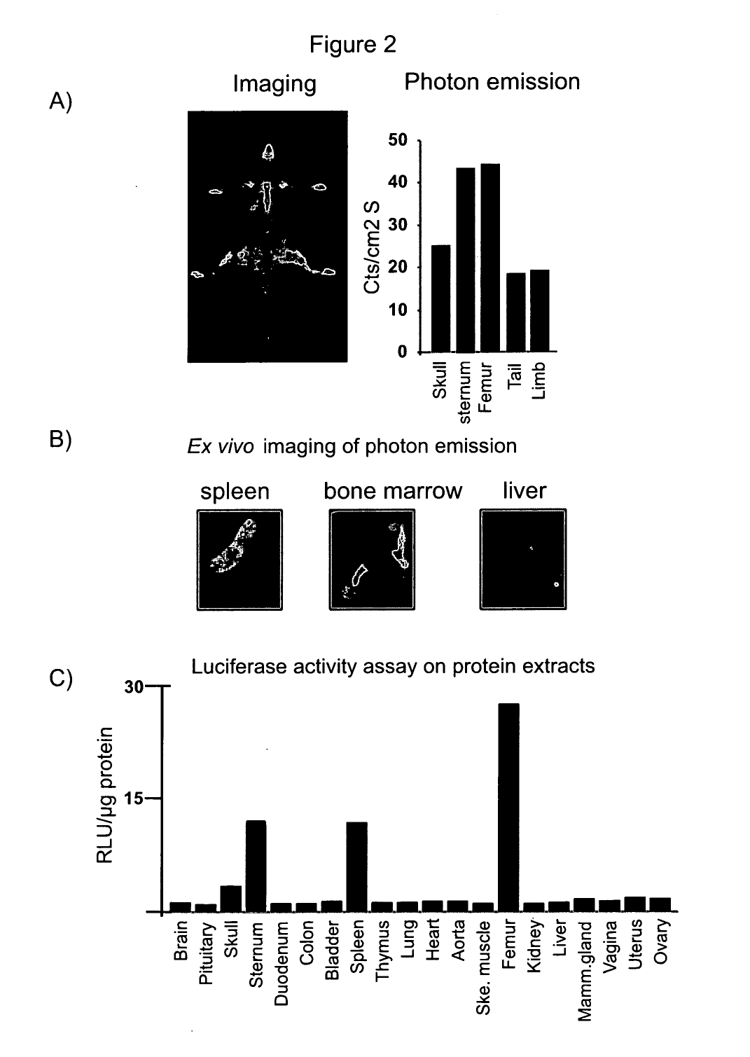

FIGS. 2 to 4, 6 and 9 show the imaging results obtained in adult transgenic animals, with related diagrams as described below.

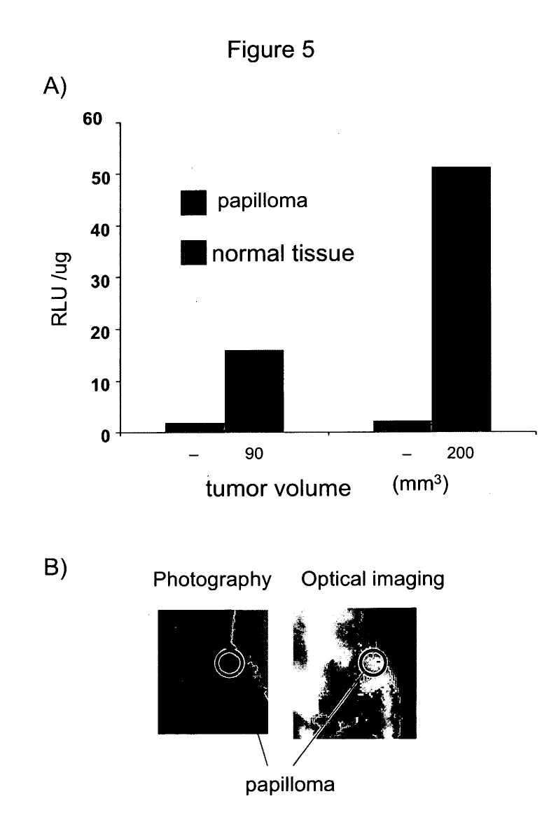

FIGS. 5, 7 and 8 show further diagrams as described below.

### EXPERIMENTAL PART

FIG. 1 shows a diagram of the construct consisting of the nucleotide sequence used for transgenesis in order to obtain an animal line of the invention, which is preferably a mouse line, consisting of a reporter system composed of a gene coding the enzyme firefly luciferase (Photinus pyralis), corresponding to SEQ ID NO:2, under transcriptional control of a 312 by fragment derived from the promoter of the gene encoding cyclin B2 (SEQ ID NO: 1), identified by the acronym CYCB2. The transgene was constructed, linearized, separated from plasmid sequences and microinjected into fertilized eggs of C57BI/6xDBA/2 using standard protocols. A total of 160 microinjected zygotes were implanted in the oviducts of 6 recipient mice that gave birth to 43 pups. To identify founder animals, genomic DNA was extracted from tail biopsies of the 43 animals and genotypic analysis was performed by PCR with oligonucleotide primers specific for the sequence of the transgene. Tail fragments were lysed overnight at 50° C. in buffer containing proteinase K and DNA was purified by the standard procedure based on extraction with phenol, chloroform and alcohol (Sambrook et al, 1989). The PCR reaction was performed using oligonucleotides with sequence:

(SEQ ID NO: 5)

| | |
|---|---|
| forward oligo | 5′TGTAGACAAGGAAACAACAAAGCCTGGTGGCC |

(SEQ ID NO: 6)

| | |
|---|---|
| reverse oligo: | 5′GGCGTCTTCCATTTTACCAACAGTACCGG |

Of 43 individuals analyzed, two were identified as carriers of the transgene, these founders were crossed with wild-type mice and the heterozygous offspring was genotyped by PCR as described above.

### Characterization and Validation of the Model

The tissues of the transgenic mouse according to the invention express luciferase only in actively proliferating cells.

The luciferase activity was monitored in animal and embryo tissues by bioluminescence imaging or by determination of enzymatic activity in protein extracts (Biserni A, Giannessi F, Sciarroni A F, Milazzo F M, Maggi A, Ciana P, In vivo imaging reveals selective peroxisome proliferator activated receptor modulator activity of the synthetic ligand 3-(1-(4-chlorobenzyl)-3-t-butylthio-5-isopropylindol-2-yl)-2,2-dimethylpropanoic acid (MK-886). Mol Pharmacol. 2008 May; 73(5):1434-43).

Bioluminescence imaging experiments on alive animals were conducted in heterozygous males 2-4 months of age. Mice were anesthesized by subcutaneous injection ketamine/xylazine and were administered one intraperitoneal dose of luciferin substrate (50 ug/kg). Fifteen minutes after luciferin injection (Biserni et al., 2008), the luminescence emitted by the animals was measured by a CCD camera (Berthold Technologies, Wilbad) and superimposed with images in reflected light to locate the emission sources. As shown in FIG. 2A, where the result of imaging is shown on the left and the photon emission graph is on the right, in the adult the distribution of photon emission is selective in some areas of the body. In particular, the emission is detected in the area corresponding to the chest (sternum), posterior limbs (femur) and areas of bone remodeling in the feet The identity of the source of light emission was confirmed by removing the organs beneath the signal and ex vivo measurement of light emission (FIG. 2B). Finally, imaging data were confirmed by the analysis of enzyme activity in protein extracts, according to the graph shown in FIG. 2C.

For this purpose, organs were homogenized in lysis buffer and lysates were subjected to three cycles of freezing and thawing. Proteins were separated from DNA and lysosomes by centrifugation (13000×g for 30 min). After determining the protein concentration of the extract, the biochemical assay of luciferase activity was carried out by measuring luminescence emission with a luminometer. The relative luminescence units (RLU) determined during a measurement of 10 seconds were then expressed on the Y axis as RLU values per microgram protein. The results according to the graph in FIG. 2C indicate that, in the adult animal, light emission is restricted to organs characterized by basal homeostatic proliferation such as spleen, bone marrow, femur and sternum, or sites of active tissue remodeling in the mouse, such as joints of the feet, jaw, etc.

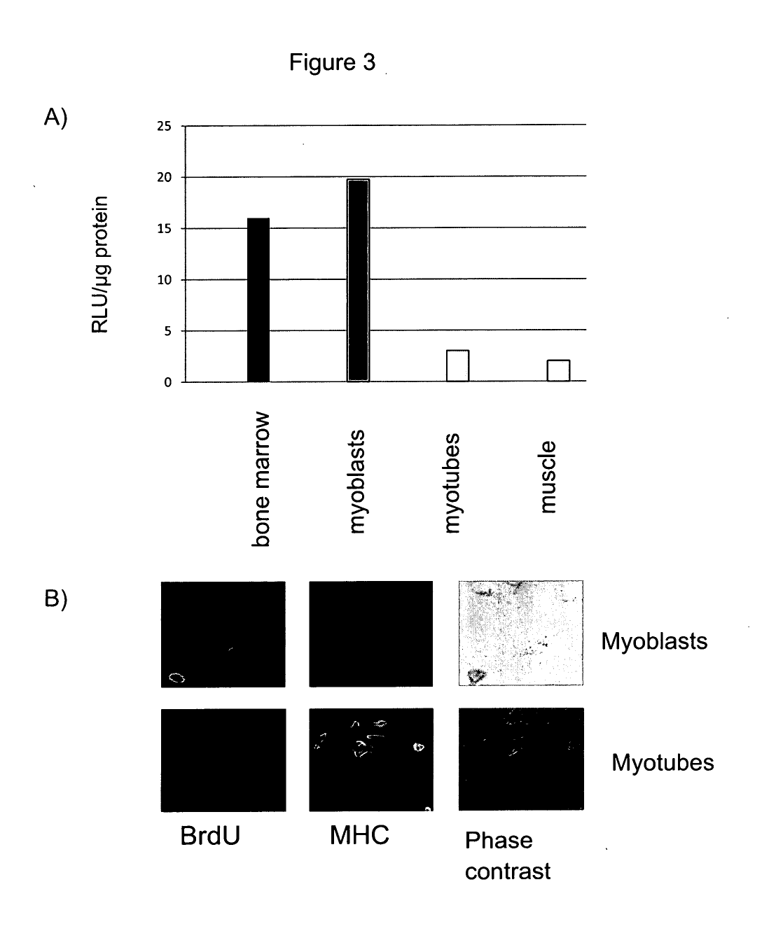

To assess the expression of luciferase in proliferating progenitor cells, bone marrow cells, that are highly enriched in hematopoietic precursors, were collected by flushing the femurs with culture medium and enzyme activity was analyzed in protein extracts (Ciana et al 2001), as shown in the graph in FIG. 3A. From these data it can be concluded that, in the transgenic mouse according to the invention, luciferase is expressed in actively proliferating precursor cells.

Another actively proliferating histological type of precursor cell is represented by muscle satellite cells grown in the presence of growth factors. Muscle satellite cells were obtained by previously described protocols: Gurtner A, Fuschi P, Magi F, Colussi C, Gaetano C, Dobbelstein M, Sacchi A, Piaggio G. NF-Y dependent epigenetic modifications discriminate between proliferating and postmitotic tissue. PLoS One. 2008 Apr. 23; 3(4):e2047. Rando T A, Blau H M, Primary mouse myoblast purification, characterization, and transplantation for cell-mediated gene therapy. J Cell Biol. 1994 June; 125(6):1275-87.

Cells were obtained from muscles of the front and hind limbs of newborn mice at 20 days of life. After removing bones, the muscle mass was subjected to enzymatic and mechanical dissociation. The resultant suspension was filtered and then centrifuged to pellet cells in suspension. The cell pellet was resuspended in culture medium enriched with growth factors (20% bovine serum, FGF, 3% embryo extract from fertilized eggs) and seeded in culture dishes to induce proliferation (myoblasts). The same cells were also seeded in collagen-containing plates and grown in culture medium devoid of growth factors (without serum) to induce muscle differentiation (myotubes). In addition, cytosine arabinoside, at a concentration of 50 μM, was added to the culture medium without serum to eliminate undifferentiated cells still proliferating. As demonstrated by the analysis of the enzymatic activity present in protein extracts, myoblasts express high levels of luciferase. In contrast, the expression of the enzyme in myotubes is reduced in the assay to undetectable levels similar to those found in differentiated muscle tissue (FIG. 3A). Myoblasts used in this essay are highly proliferating, as demonstrated by their ability to incorporate bromodeoxyuridine (BrdU), a thymine analog that is incorporated in newly synthesized DNA, hence a marker to identify actively proliferating cells. In immunofluorescence assays, antibodies against BrdU were used to label cells that incorporated BrdU. In contrast myotubes do not incorporate BrdU, thus demonstrating that they are not proliferating. In addition, as expected, proliferating myoblasts do not express markers typical of differentiated muscle cells, such as MHC (Myosin Heavy Chain). The myotube differentiation index is high, as demonstrated by MHC expression shown in FIG. 3B.

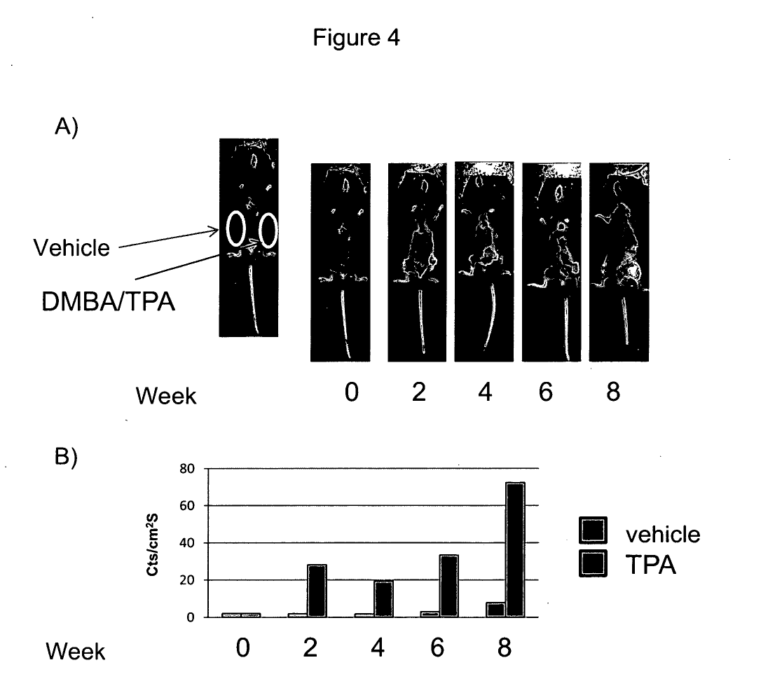

To further demonstrate that in the transgenic mouse according to the invention luciferase expression is correlated with cell proliferation, we applied a protocol that was used to induce skin hyperproliferation and thereby skin papillomas. The protocol involves cutaneous application of 0.24% 7,12-dimethylbenz(a)anthracene (DMBA) solution in acetone for a week, and of 5 nmoles of 12-O-tetradecanoylphorbol-13-acetate (TPA) weekly for a time sufficient to obtain palpable skin papillomas (about 3 months). Tumor promoters were applied to the ventral right region, while only the vehicle was applied to the left area; animals were subjected to the imaging procedure every two weeks up to 8 weeks, obtaining the results shown in FIG. 4A. Increased light emission is observed in the area treated with DMBA and TPA, while the emission of photons remains roughly constant over time in the contralateral area treated with vehicle alone. The graph in FIG. 4B shows how the intensity of the effect varies over time. In agreement with these findings, the protein extracts from tumors that developed after treatment have a significantly higher luciferase activity compared to the normal tissue, as shown in FIG. 5. In the graph in FIG. 5A, it is interesting to note that the relative amount of luciferase is higher in the larger tumor compared to the smaller tumor; this difference apparently reveals that a larger amount of proliferating cells are present in the larger tumor. FIG. 5B shows the presence of the papilloma, both by photography and optical imaging.

### Ubiquitous Activity

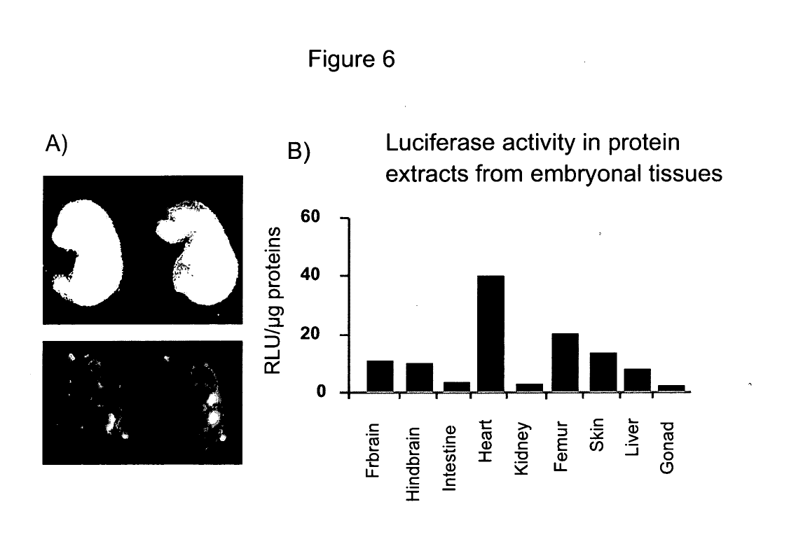

Finally, luciferase expression was analyzed in mouse embryos at the stage of 19 days post-conception, where cells of all tissues show a strong proliferative activity. Expression of the reporter gene was assessed by ex vivo imaging analysis performed on the whole embryo, shown in FIG. 6A. For this purpose, wild type C57/BI6 pregnant females impregnated by transgenic males were treated with a single intraperitoneal injection of 150 ug/kg luciferin; 15 minutes later the animal was sacrificed and embryos were subjected to bioluminescence analysis. As shown in FIG. 6B, all embryonic tissues analyzed, as specified therein, show a generalized light emission, a conclusion proven by an analysis of luciferase activity carried out on all tissues that could be isolated by micro-dissection.

Thus, the biosensor is active in all proliferating cells.

### Activity of Compounds Modulating Proliferation

The reporter mouse generated according to the invention, as described above, is used as a tool to evaluate natural or synthetic molecules with pro- or anti-proliferative activity. Therefore the reporter mouse enables simultaneous quantification of the activity of a compound on cell proliferation in all tissues and in the physiological context. The method can be applied to:

1) development of compounds of pharmacological interest inhibiting cellular proliferation, such as antitumor compounds, anti-inflammatory compounds, etc.
2) development of compounds of pharmacological interest inducing cell proliferation, such as growth factors that selectively stimulate stem cell proliferation.
3) toxicological evaluation of compounds with potential tumorigenic activity, as well as assessment of compounds with chemopreventive anticancer activity.

### 1) Activity of Compounds Inhibiting Proliferation

As an example of application for development of anti-proliferative drugs, heterozygous individuals of the transgenic mouse line according to the invention were treated as follows: a first group was treated with 150 ug/kg 5-fluorouracil (5FU), a well-known chemotherapeutic agent, while another group was treated with saline as control (vehicle) for 2 and 5 days.

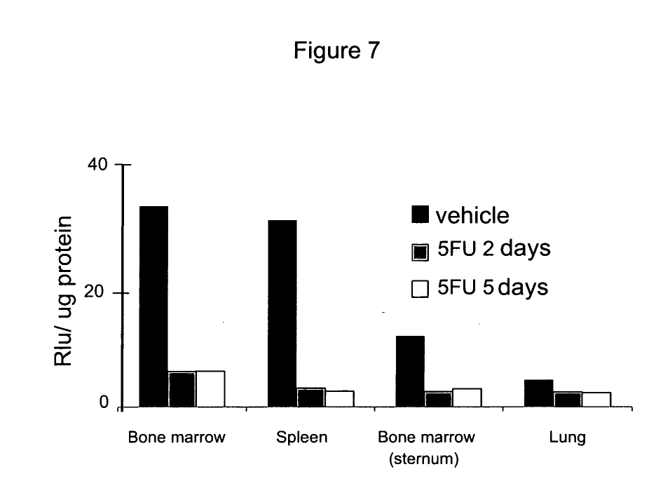

Mice were sacrificed and luciferase activity was evaluated in tissue protein extracts, as reported in the graph in FIG. 7. Reduced luciferase activity, until levels that were undetectable by the method of analysis, were found in all the tissues of 5FU treated mice that were subjected to analysis.

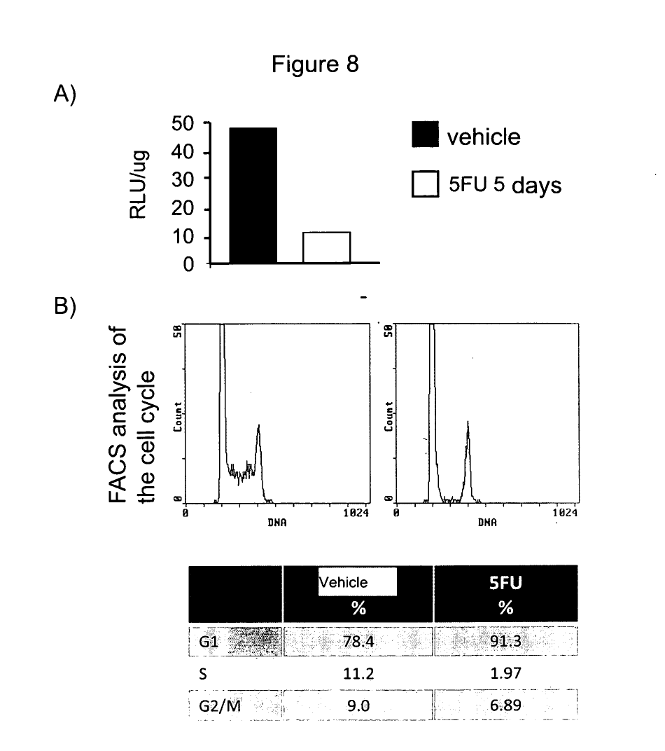

To demonstrate that the reduction of luciferase was related to reduced proliferation, bone marrow cells from mice treated with vehicle or 5FU were analyzed by detecting the cell cycle phase, using a methodology based on flow cytometry (FACS) and cell staining with propidium iodide. Cells were fixed with methanol acetone 4:1, stained with 0.1 mg/ml propidium iodide, a DNA fluorescent marker, and then subjected to FACS analysis. The graph in FIG. 8B shows that 5FU treatment induces a cell cycle block indicated by a decrease to 1.97% of S phase cells compared to a corresponding value of 11.2% detected in the sample treated with vehicle alone. These data demonstrate that it is possible to quantify the activity of compounds that reduce cell proliferation in tissues of the transgenic mouse according to the invention.

Data in the graph representing RLUs as function of microgram protein, in FIG. 8A, demonstrate that a 5 day 5FU treatment inhibited proliferation and correspondingly reduced luciferase expression also in bone marrow cells.

### 2) Activity of Compounds Inducing Proliferation

As discussed above, the data shown in FIG. 3 on luciferase expression in proliferating progenitor cells and not in differentiated cells are an example of the application of the reporter mouse according to the invention to evaluate the action of growth factors on normal progenitor cells. As shown by the experiment, precursor stem cells placed in medium enriched with growth factors maintain stem cell properties, proliferate and express luciferase, whereas, if these precursors are placed in differentiation medium, cells differentiate, undergo cell cycle arrest and correspondingly lose the expression of the reporter.

3) Toxicological Evaluation of Compounds with Potential Tumorigenic Activity

A further application of the transgenic animal according to the invention is in the toxicological field. In particular, the ability to detect in tissues of the transgenic animal according to the invention a potential proliferative activity of a given compound, alone or in combination with other compounds, makes possible to delineate a map of the potential pro-carcinogenic activity.

To evaluate this application, the skin of a transgenic mouse according to the invention was treated with DMBA, a carcinogen, and the stimulation of light emission was measured for the carcinogen-treated side compared to the side treated with vehicle alone.

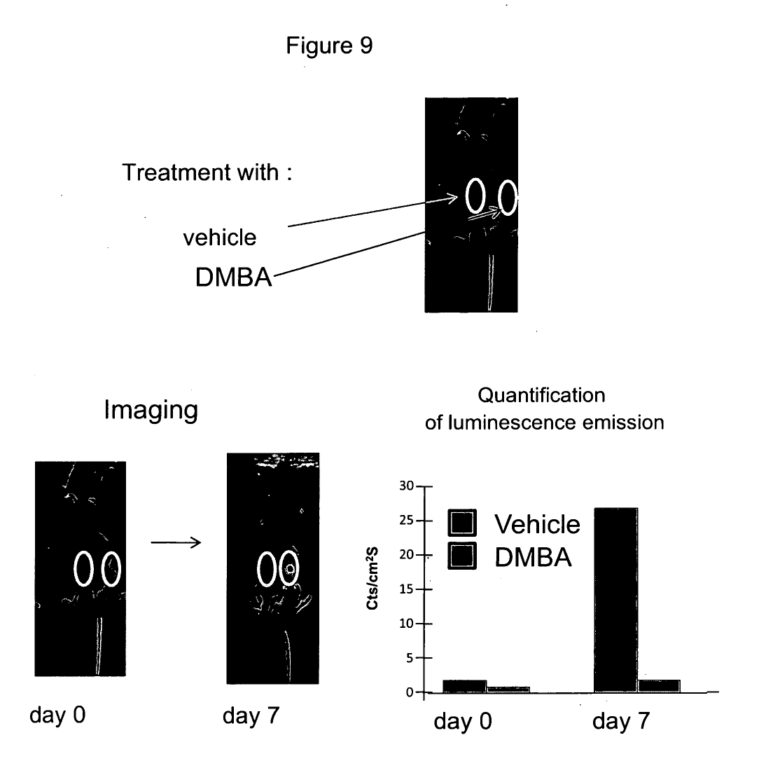

In particular, a 0.24% DMBA solution in acetone was applied to the skin of transgenic individuals according to the invention. One week later (day 7 relative to day 0) the amount of luciferase produced was detected by measuring the light emission produced by the skin of the animals. FIG. 9 shows the corresponding imaging data.

The graph in FIG. 9 shows a 12.5 fold induction of luciferase activity in the region of absorption of the compound compared to the bilateral region treated with vehicle alone, thus showing that the biosensor can detect the activity of carcinogens at an early stage, long before the development of a full-blown cancer. From the overall description given above, it is concluded that the transgenic animal according to the present invention provides an effective method to delineate the full profile of pharmacological activity of modulators of cell proliferation, through a simple and direct measurement of the proliferative state of animal tissues, both under physiological conditions and following pharmacological treatment.

1) Transgenic, non-human mammalian animal whose genome incorporates, as a biosensor of the activity of cellular proliferation modulators, a DNA sequence comprising the luciferase reporter transgene ligated downstream of a sequence of the CYCB2 gene promoter, said promoter/reporter sequence being flanked by HS4 insulator sequences at each of its 3′ and 5′ sides.

2) Animal according to claim 1, characterized in that it is a mouse.

3) Use of the transgenic non-human mammal animal according to claim 1, for studying and developing compounds provided with an inhibitory activity on cellular proliferation and which are of potential pharmaceutical interest.

4) Use according to claim 3, for studying and developing compounds provided with anti-tumor activity.

5) Use according to claim 3, for studying and developing compounds provided with anti-inflammatory activity.

6) Use of the transgenic non-human mammal animal according to claim 1, for studying and developing compounds provided with an inducing activity on cellular proliferation and which are of potential pharmaceutical interest.

7) Use according to claim 6 for studying and developing compounds which are of potential pharmaceutical interest as growth factors stimulating selective proliferation of stem cells.

8) Use of the transgenic non-human mammal animal according to claim 1, for studying and for toxicologically evaluating compounds provided with tumorigenic activity.

9) Use of the transgenic non-human mammal animal according to claim 1, for studying and evaluating compounds provided with chemo-preventive activity against tumor insurgence.

10) Method for evaluating the activity of a cellular proliferation modulator according to claim 1, characterized in that it comprises the steps of: constructing a transgenic non-human mammal animal line by incorporating in its genome a DNA sequence comprising the luciferase reporter transgene ligated downstream of a sequence of the CYCB2 gene promoter, said promoter/reporter sequence being flanked by HS4 insulator sequences at each of its 3′ and 5′ sides; treating individuals of said transgenic animal line, or tissues or cells derived therefrom, with said cellular proliferation modulator; comparing the luminescence emission in individuals treated with said cellular proliferation modulator and in other non-treated individuals of the same transgenic animal line.

11) Method according to claim 10 for evaluating the inhibitory activity on cellular proliferation, in particular on anti-tumor activity or anti-inflammatory activity, characterized in that it comprises the steps of: treating living individuals of said transgenic animal line, or tissues or cells derived therefrom, with said cellular proliferation modulatory agent; evaluating the amount of luciferase produced by the animal by in vivo imaging assays, or produced by its tissues and by its cells by ex vivo enzymatic activity assays; comparing the luminescence emission in individuals treated with said cellular proliferation modulatory agent and in other non-treated individuals of the same transgenic animal line.

12) Method according to claim 10 for evaluating the inducing activity on cellular proliferation, in particular the activity of compounds stimulating selective proliferation of stem cells, characterized in that it comprises the steps of: treating living individuals of said transgenic animal line, or tissues or cells derived therefrom, with said cellular proliferation modulatory agent; evaluating the amount of luciferase produced by the animal by in vivo imaging assays, or produced by its tissues and by its cells by ex vivo enzymatic activity assays; comparing the luminescence emission in the individuals treated with said cellular proliferation modulator and in other non-treated individuals of the same transgenic animal line.

13. Method according to claim 10 for evaluating the tumorigenic activity characterized in that it comprises the steps of: treating living individuals of said transgenic animal line, or tissues or cells derived therefrom, with said cellular proliferation modulatory agent; evaluating the amount of luciferase produced by the animal by in vivo imaging assays, or produced by its tissues and by its cells by ex vivo enzymatic activity assays; comparing the luminescence emission in the individuals treated with said cellular proliferation modulator and in other non-treated individuals of the same transgenic animal line.

14. Method according to claim 10, for evaluating the chemo-preventive activity against tumor insurgence, characterized in that it comprises the steps of: treating living individuals of said transgenic animal line, or tissues or cells derived therefrom, with said cellular proliferation modulatory agent; evaluating the amount of luciferase produced by the animal by in vivo imaging assays, or produced by its tissues and by its cells by ex vivo enzymatic activity assays; comparing the luminescence emission in the individuals treated with said cellular proliferation modulator and in other non-treated individuals of the same transgenic animal line.


SEQUENCE LISTING (6 sequences)

SEQ ID NO: 1 - DNA, 312 bp, Mus musculus
  promoter (1)..(312): Cyclin B2 GenBank No. AY88998
tatgtccttaggtgatggctccccactcctacccctaaaattaagacaatcttttccagtaggaggaaaaattattttatttaatatcagggactagaatttgaaaataagactgtagacaaggaaacaacaaagcctggtggcctcgctggttgctatgacaagcaaatacaagccagccaatcaacgtgcagaaaggccttccagtctagccaatgggttgcgcgcgccctgcgtgcgtctacccaatagtgcgtcagcggcgcggtatttgaatcgcggaccgggcggtggacgcggagcggcggggcc

SEQ ID NO: 2 - DNA, 1653 bp, Photinus pyralis
atggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaa

SEQ ID NO: 3 - protein, 550 aa, Photinus pyralis
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 4 - DNA, 2392 bp, Gallus gallus
  misc_feature (1)..(2392): Insulator sequence from b-globin-gene GenBank No. AJ277959
agggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctgggggatacggggaaaaagctttaggctgaaagagagatttagaatgacagaatcatagaacgcctgggttgcaaaggagcacagtgctcatccagatccaaccccctgctatgtgcaggtcatcaaccagcagcccagcgcgtcagagccacatccagcctggccttgaatgcctgcctgcagggatggggcatccacagcctccttgggcaacctgttcagtgcgtcaccaccctctggggaaaaactgcctcctcatatccaacccaaacctcccctgtctcagtgtaaagccattcccccttgtcctatcaagggggagtttgctgtgacattgttggtctggggtgacacatgtttgccaattcagtgctcacggagaggcagatcttgggataaggaagtgcaggacagcatggacgtggacatgcaggtgttgaggctctggacactccaagtcacagcgttcagaacagccttaaggtcaagaagataggatagaaggacaaagagcaagttaaaacccagcatggagaggagcacaaaaaggccacagacactgctggtccctgtgtctgagcctgcatgtttgatggtgtctggatgcaagcagaaggggtggaagagcttgcctggagagatacaggctgggtcgtaggactgggacaggcagctggagaattgccatgtagatgttcatacaatcgtcaaatcatgaaggctggaaaagctccaagatccccaagaccaaccccaacccacccaccgtgccactggccatgtccctcagtgccacatccccacagttcttcatcacctccagggacggtgaccccctcctccgtggcagctgtgccactgcagcaccgctctttggagaaggtaaatcttgctaaatccagcccgaccctcccctggcacaacgtaaggccattatctctcatccaactccaggacggagtcagtgagaatatttctagagagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctgggggatacggggaaaaagctttaggctgaaagagagatttagaatgacagaatcatagaacgcctgggttgcaaaggagcacagtgctcatccagatccaaccccctgctatgtgcaggtcatcaaccagcagcccagcgcgtcagagccacatccagcctggccttgaatgcctgcctgcagggatggggcatccacagcctccttgggcaacctgttcagtgcgtcaccaccctctggggaaaaactgcctcctcatatccaacccaaacctcccctgtctcagtgtaaagccattcccccttgtcctatcaagggggagtttgctgtgacattgttggtctggggtgacacatgtttgccaattcagtgctcacggagaggcagatcttgggataaggaagtgcaggacagcatggacgtggacatgcaggtgttgaggctctggacactccaagtcacagcgttcagaacagccttaaggtcaagaagataggatagaaggacaaagagcaagttaaaacccagcatggagaggagcacaaaaaggccacagacactgctggtccctgtgtctgagcctgcatgtttgatggtgtctggatgcaagcagaaggggtggaagagcttgcctggagagatacaggctgggtcgtaggactgggacaggcagctggagaattgccatgtagatgttcatacaatcgtcaaatcatgaaggctggaaaagctccaagatccccaagaccaaccccaacccacccaccgtgccactggccatgtccctcagtgccacatccccacagttcttcatcacctccagggacggtgaccccctcctccgtggcagctgtgccactgcagcaccgctctttggagaaggtaaatcttgctaaatccagcccgaccctcccctggcacaacgtaaggccattatctctcatccaactccaggacggagtcagtgagaatatt

SEQ ID NO: 5 - DNA, 32 bp, Mus musculus
tgtagacaaggaaacaacaaagcctggtggcc

SEQ ID NO: 6 - DNA, 29 bp, Artificial Sequence
  Synthesized; PCR Primer
ggcgtcttccattttaccaacagtaccgg